(19) United States
(12) Patent Application Publication
(10) Pub. No.: US 2020/0337680 A1
(43) Pub. Date: Oct. 29, 2020

(54) SYSTEMS AND METHODS FOR LOCATING AND MONITORING A SUBCUTANEOUS TARGET SITE

(71) Applicant: THE BOARD OF TRUSTEES OF THE LELAND STANFORD JUNIOR UNIVERSITY, STANFORD, CA (US)

(72) Inventors: Dimitri Alain Augustin, Los Altos, CA (US); Racquel Tiffany Redwood, Cincinnati, OH (US); Zachary Andrew Wolf, Mountain View, CA (US); Bryan Irby Hartley, Redwood City, CA (US)

(21) Appl. No.: 16/857,131

(22) Filed: Apr. 23, 2020

Related U.S. Application Data
(60) Provisional application No. 62/839,931, filed on Apr. 29, 2019.

(51) Int. Cl. A61B 8/08; A61B 8/00

(52) U.S. Cl. CPC A61B 8/488; A61B 8/0858; A61B 8/461

(57) ABSTRACT

Devices and methods are provided herein for locating and monitoring subcutaneous target sites. A device as described herein may comprise a base layer configured to attach to a portion of a subject's body in proximity to the target site; and a plurality of transducer elements on the base layer, wherein the plurality of transducer elements are configured to (1) transmit a set of signals that penetrate beneath the skin of the subject and (2) receive a set of reflected signals associated with underlying anatomical structures, the underlying anatomical structures comprising the target site and bodily tissue surrounding the target site, wherein the set of reflected signals is used to identify a location of the target site while the device is placed in situ on the subject's body, without having to physically move the device over the portion of the subject's body to search for the target site.

### RELATED APPLICATION DATA

The present application claims benefit of co-pending provisional application Ser. No. 62/839,931, filed Apr. 29, 2019, the entire disclosure of which is expressly incorporated by reference herein.

### BACKGROUND

End stage renal disease affects over 700,000 patients in the United States. Most of these patients must undergo frequent hemodialysis, which involves removal and filtering of the patient's blood in a hemodialysis machine. To receive hemodialysis, there must be a way to frequently and effectively access the patient's circulatory system with a needle. This is often achieved by a surgeon creating an arteriovenous (AV) fistula on the patient's arm. An AV fistula involves connecting an artery to a vein, so the fistula becomes larger and is thus easy to see and puncture with a needle.

Unfortunately, surgically created AV fistulas fail as often as 50% of the time, according to some studies. One of the biggest challenges with AV fistulas is effectively monitoring them, to know when they are failing. The average time a patient has to wait to first use a fistula after it is surgically made on the arm is over three months.

Current systems for dialysis access surveillance are limited. Current Doppler ultrasound techniques are expensive, require a trained operator and are time consuming. Other methods require the patient to be connected to an extracorporeal hemodialysis system. Patients with new fistulas or grafts that are not being used cannot benefit from these extracorporeal surveillance systems.

Therefore, it would be advantageous to provide clinicians with a portable, low-cost method for dialysis access surveillance, without requiring an advanced operator or connection to an extracorporeal system that enhances patient compliance.

### SUMMARY

The present disclosure addresses the above limitations. Various embodiments of the present disclosure address the demand for accessible, easy to use monitoring systems that are capable of locating and monitoring a subcutaneous target site without the need for a skilled operator or the need to be connected to an extracorporeal hemodialysis system.

According to some aspects of the disclosure, a device for locating and monitoring a subcutaneous target site is provided. The device may comprise a base layer configured to attach to a portion of a subject's body in proximity to the target site; and a plurality of transducer elements on the base layer, wherein the plurality of transducer elements are configured to (1) transmit a set of signals that penetrate beneath the skin of the subject and (2) receive a set of reflected signals associated with underlying anatomical structures, the underlying anatomical structures comprising the target site and bodily tissue surrounding the target site, wherein the set of reflected signals is used to identify a location of the target site while the device is placed in situ on the subject's body, without having to physically move the device over the portion of the subject's body to search for the target site. In some embodiments, the device may be capable of being used or operated stand-alone, without requiring the subject to be connected to an extracorporeal hemodialysis system. The device may be capable of being used or operated by the subject without requiring assistance from a healthcare technician.

In some cases, the device may comprise a power source and a transmitter. The power source may comprise a battery. In some embodiments, the device may further be configured to operably couple to a power adapter for charging the battery before or after usage of the device. The device may further comprise a processor. In some embodiments, the processor may be configured to generate a set of image data from set of reflected signals. In some cases the set of image data may comprise color Doppler image data, 2D ultrasound image data or any other ultrasound image data.

Also disclosed herein is a method of locating a surgically created subcutaneous target site, the method comprising: attaching a device to a portion of the subject's body in proximity to the target site, wherein the device comprises a base layer and a plurality of transducer elements on the base layer; using the plurality of transducer elements to (1) transmit a set of signals that penetrate beneath the skin of the subject and (2) receive a set of reflected signals associated with underlying anatomical structures, the underlying anatomical structures comprising the target site and bodily tissue surrounding the target site; and analyzing the set of reflected signals to identify a location of the target site while the device is placed in situ on the subject's body, without having to physically move the device over the portion of the subject's body to search for the target site.

In some embodiments, a subcutaneous target site is created via open surgery or percutaneously. Examples of subcutaneous target sites include but are not limited to arteriovenous fistulas, arteriovenous grafts, or arteriovenous shunts or any type of connections used for hemodialysis.

In some embodiments, the base layer is flexible and configured to substantially conform to the portion of the subject's body. In some cases the base layer is attached to the portion of the subject's body using an adhesive, or a strap, and/or a conductive medium that permits the transmitted signals and received signals to pass through. The portion of the subject's body may include an upper extremity or a lower extremity. The base layer can serve as a substrate for supporting the plurality of transducer elements. The base layer may comprise a plurality of wires for electrically connecting the plurality of transducer elements. In some embodiments, the plurality of transducer elements are disposed within the base layer.

In some embodiments, the plurality of transducer elements are selected from the group consisting of but not limited to ultrasound sensors, acoustics sensors, optical sensors, pressure sensors, strain sensors, vibration sensors, pulse oximeters, and thermal sensors. The plurality of transducer elements can be arranged in a one-dimensional longitudinal array or a two-dimensional grid array to form one or more detection channels. In some embodiments, the plurality of transducer elements are spaced apart by a distance ranging from micron level to millimeter level. In some instances, the plurality of transducer elements are spaced laterally apart by a first distance ranging from micron level to millimeter level, and spaced longitudinally apart by a second distance ranging from micron level to millimeter level. The two-dimensional grid array may comprise an m by n array, wherein m and n are same or different integers. At least m or n may be greater than 2. The two-dimensional grid array may comprise a rectangular array or a square array.

In some embodiments, the set of reflected signals comprises one or more pulse waves. In some cases, the processor is configured to generate image data of the one or more pulse waves. The image data may be based on at least two pulses/heartbeats. The image data may comprise an average velocity over about 4 to 8 heartbeats in about 4 seconds.

In some cases, the set of reflected signals display different signal intensities and actively change over time depending on characteristics of the underlying anatomical structures. In some cases, magnitudes of the reflected signals are indicative of the signal intensities. The plurality of transducer elements may be operably coupled to a processor via wired or wireless communications.

In some embodiments, the device further may comprise a processor. The processor may be provided separate from the device. In some embodiments, the processor is located on a mobile device or an external monitoring unit. The processor may be configured to receive the set of reflected signals from the plurality of transducer elements. The processor may further be configured to identify the location of the target site by comparing the different signal intensities within the set of reflected signals. In some embodiments, the processor is configured to identify the location of the target site based on a spatial configuration of a first set of transducer elements having the highest reflected signal intensity. The first set of transducer elements having the highest reflected signal intensity may be in a straight or linear spatial configuration. The first set of transducer elements having the highest reflected signal intensity may be in a staggered or non-linear spatial configuration.

In some embodiments, the processor is configured to use the first set of transducer elements to further monitor the state of the target site. The state of the target site may comprise a maturity, function and/or dysfunction of the target site. The state of the target site may be indicated by one or more physiological parameters selected from the group consisting of blood flow within vessel, depth from surface of the skin to the vessel wall, diameter of the vessel, velocity of blood within vessel, compliance of vessel and thickness of vessel wall. The one or more physiological parameters may be associated with an arteriovenous fistula, an arteriovenous graft, an arteriovenous shunt, any type of connection used for hemodialysis, veins, arteries, capillaries, ducts, or any other anatomical conduits.

In some embodiments, the plurality of transducer elements comprise (1) the first set of transducer elements and (2) a second set of transducer elements having lower reflected signal intensities than the first set of transducer elements. In some cases, the processor is not configured to use any of the reflected signals from the second set of transducer elements when monitoring the state of the target site. The processor may be configured to discard or disregard the reflected signals from the second set of transducer elements after the location of the target site has been identified. In some embodiments, the processor is configured to deactivate the second set of transducer elements once the location of the target site has been identified. The second set of transducer elements may further be configured to (1) cease transmitting signals and (2) cease receiving reflected signals once the location of the target site has been identified.

In some embodiments, the plurality of transducer elements are disposed at a fixed or variable angle relative to the skin or the target site of the subject. The angle may range from about 40 degrees to 60 degrees.

In some embodiments, the processor may be configured to monitor the state of the target site based on a plurality of measurements taken over a time period. The plurality of measurements may comprise a baseline measurement and a series of interval measurements. The time period may be on the order of hours, days, weeks, or months.

In some embodiments, processor is configured to measure the maturity, function and/or dysfunction of the target site, by comparing the series of interval measurements to the baseline measurement. The maturity, function, and/or dysfunction may be measured based at least on blood flow volume, diameter of blood vessel, or depth of the vessel.

In some embodiments, the processor may be configured to determine whether the target site is (1) progressing towards maturation or (2) digressing from maturation, based on the comparisons between the series of interval measurements to the baseline measurement. The processor may be further configured to determine whether the target site is (1) maintaining function when the target site is mature or (2) digressing from function which is indicative of the target site failing, based on the comparisons between the series of interval measurements to the baseline measurement.

In some embodiments, the processor may be configured to generate one or more intervention alerts to a healthcare provider or the subject (patient), based on the state of the target site. The one or more intervention alerts may be designed to allow the healthcare provider or the subject (patient) to take corrective actions to further address a potential adverse occurrence at the target site.

In some embodiments, the target site is capable of being located using the device, and without requiring a technician to obtain ultrasound image scans and use digital calipers on the displayed image scans to measure and find the target site.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1C show a perspective view of a portion of the body at the upper extremity with a flow monitoring device, according to one embodiment;

FIG. 1D shows a perspective view of an arm with device and fastening mechanism;

FIGS. 2A and 2B show a side view and cross section of an arm with the device laid on top of the arm comprising multiple transducer elements and their respective transmitting pulses (1, 2 and 3);

FIG. 2C shows three reflected pulse waves with respect to the three transmitted pulses in FIGS. 2A and B;

FIG. 3A is a perspective view of a diagram of a single array device;

FIG. 3B is a perspective view of a diagram of a multi-array device;

FIG. 4A is a perspective view of an arm with attachable and/or detachable device;

FIG. 4B is a perspective view of an arm with a device and device hub;

FIG. 4C is a perspective view of an arm with a device in separate parts;

FIG. 5 is a side view of the arm with a device with a conformable base layer;

FIG. 6 is a transverse plane view of the arm with device with conformable base layer;

FIGS. 7A-7F show an illustration of spatial configuration of device relative the target site and signal intensities in different spatial configurations;

FIG. 8 is a block diagram for a method of capturing data and processing data;

FIG. 9 is a perspective view of a flow diagram of an exemplary system;

FIG. 10 shows an exemplary embodiment of a process of creating an AV fistula, monitoring an AV fistula during the maturation using the wearable device and initiation of dialysis with fistula;

FIG. 11 shows an example of data analysis to determine maturation or failure of a fistula;

FIG. 12 shows an exemplary embodiment of a smart device application output;

FIG. 13 is a perspective view of a flow diagram of care pathway;

FIG. 14 shows a schematic of an embodiment of the device on a body part;

FIG. 15 shows an example of an AV fistula and hemodialysis machine connection;

FIG. 16 shows an example of a tunneled catheter and AV fistula as well as failure time frame.

### DETAILED DESCRIPTION

Reference will now be made in detail to some exemplary embodiments of the disclosure, examples of which are illustrated in the accompanying drawings. Wherever possible, the same reference numbers will be used through the drawings and disclosure to refer to the same or like parts.

Current systems for dialysis access surveillance typically may require a trained operator to locate a target site and the use of calipers to make measurements on the ultrasound images to determine the state of a target site such as a subcutaneous AV fistula. Furthermore, the high rate of failure of surgically created AV fistulas calls for a need to have an accessible, easy to use monitoring system that can enable prediction of possible dysfunction and early intervention. The embodiments of the disclosure described herein can enable location and monitoring a subcutaneous target site without requiring a user to manually move or manually scan the device over the site. Aspects of the present disclosure may reduce or eliminate the need for a skilled operator to locate and measure the related parameters of the site. The present disclosure can be used or operated stand-alone, without requiring the subject to be connected to an extracorporeal hemodialysis system.

The present application generally relates to devices, methods and systems in the field of medical devices. In particular, it relates to devices and methods for noninvasive and/or minimally invasive, portable and/or wearable arteriovenous fistula and arteriovenous graft flow measurements. The devices and methods described herein may enable measuring subcutaneous processes, such as flow measurements, and detecting physiological phenomena in a patient's body. The present application may incorporate modalities and/or technologies, such as but not limited to a two-dimensional echo probe for detecting textures under the skin, light technology, sound and/or vibration detection, thermal dilution technology, pressure sensing technology, pulse wave technology and/or stress strain detection technology. The present application may incorporate ways of performing monitoring for humans and/or animals of all ages in various fields, including but not limited to monitoring physiological parameters in veins, arteries, capillaries, ducts, and/or other anatomical conduits.

A device as described herein can be configured to locate and/or monitor a subcutaneous target site, the device comprising a base layer configured to attach to a portion of a subject's body in proximity to the target site; and a plurality of transducer elements on the base layer, wherein the plurality of transducer elements are configured to (1) transmit a set of signals that penetrate beneath the skin of the subject and (2) receive a set of reflected signals associated with underlying anatomical structures, the underlying anatomical structures comprising the target site and bodily tissue surrounding the target site, wherein the set of reflected signals is used to identify a location of the target site while the device is placed in situ on the subject's body, without having to physically move the device over the portion of the subject's body to search for the target site. Subcutaneous target sites by way of non-limiting examples may include arteriovenous fistulas, arteriovenous grafts, arteriovenous shunts, or any other type(s) of connection used for hemodialysis. In some embodiments, the subcutaneous target site may be created via open surgery or percutaneously.

In some embodiments, ultrasound monitoring systems and components may be used, including framework systems for mounting, locating and maintaining one or more ultrasound transducer(s), or probe(s), in contact with an anatomical surface (e.g., skin) of a subject, adjustable probe mounting systems, and probe interface components providing an interface between an ultrasound probe mounting system and the probe and, optionally, providing an acoustically transmissive coupling for contacting a subject's skin or another anatomical surface. Methods for using the probe mounting systems, interface components and/or framework structure, and for adjusting the acoustic illumination area of ultrasound probes with respect to a target site are also disclosed. These aspects can optimize the quality of the measurements obtained and improve ease of use for the user or wearer. Embodiments of the disclosure may be capable of being used or operated stand-alone, without requiring the subject to be connected to an extracorporeal hemodialysis system. A device as described herein may also be capable of being used or operated by the subject in a convenient manner without requiring assistance from a healthcare technician.

The embodiments of the disclosure described herein can enable determining a state of a target site. The state of target site can comprise a maturity of the target site. The state of the target site can also include the functioning (functionality) or lack of function (dysfunction) of the site.

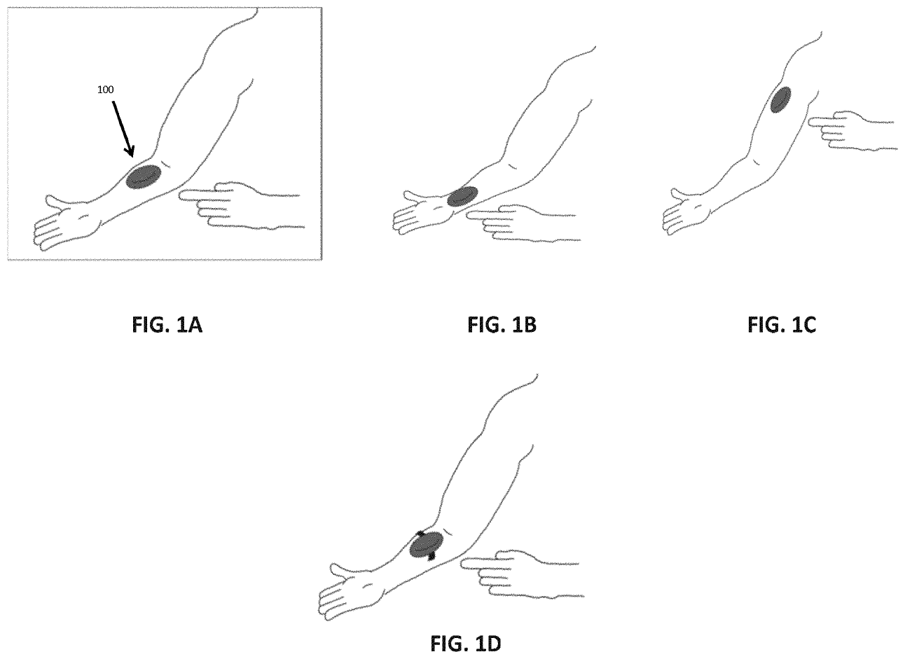

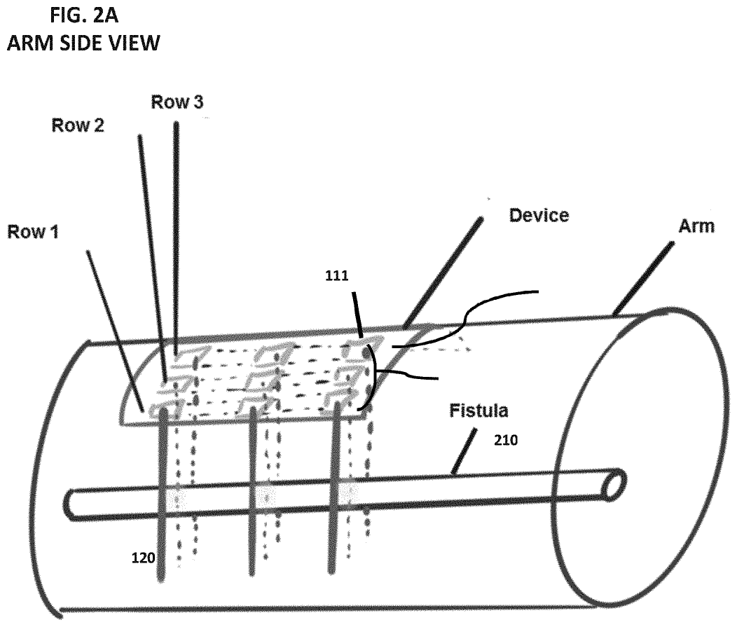

FIG. 1A illustrates an embodiment of a flow measurement device 100 placed on the upper extremity of a subject. The portion of the subject's body may include an upper extremity or a lower extremity. In various embodiments, non-invasive blood flow measurements can be obtained directly and/or indirectly through methods such as ultrasound, photoplethysmography, optical sensors, pressure sensing and/or sound. Measurement data and differences in blood flow can be stored, recorded, trended and/or analyzed. FIG. 1A, FIG. 1B, FIG. 1C and FIG. 1D illustrate embodiments of the device at different locations on the upper extremity, such that the device 100 is relative to the hemodialysis access, including the wrist, forearm and/or arm. FIG. 1D illustrates an embodiment of the device 100 that can be secured fully, partially or not at all around the body part and lay over the skin area of the hemodialysis access. Non-limiting examples of fastening and or securing attachments are straps, adhesive tapes, Velcro and other fastening mechanisms.

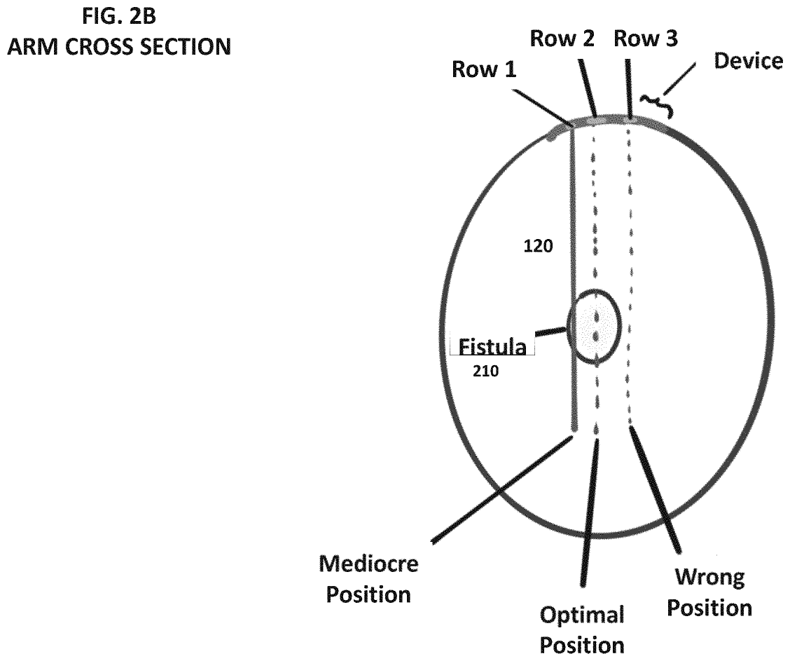

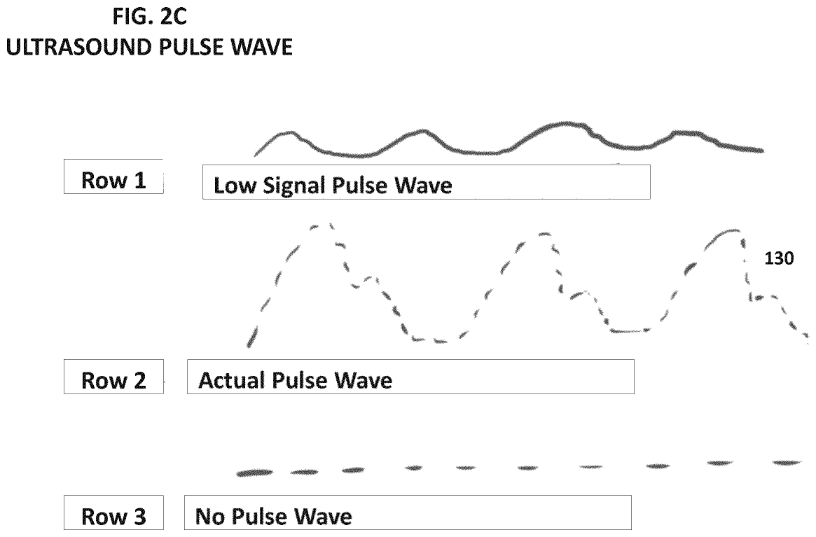

FIGS. 2A-2B show a schematic of a plurality of transducer elements 111, forming a multi-transducer array 150 on the base layer 110, overlaid on the surface of the body of subject in proximity to the target site, wherein the plurality of transducer elements may be configured to (1) transmit a set of signals 120 that penetrate beneath the skin of the subject and (2) receive a set of reflected signals 130 associated with underlying anatomical structures, the underlying anatomical structures comprising the target site and bodily tissue surrounding the target site. The bodily tissue surrounding the target site may be muscle, bone or any other bodily tissue. In some embodiments of the device, the plurality of transducer elements 111 may be disposed within the base layer 110. In some cases, the base layer may serve as a substrate for supporting the plurality of transducer elements. In some embodiments, the base layer may comprise a plurality of wires for electrically connecting the plurality of transducer elements.

FIG. 2C shows an example of reflected signals 130 relative to the location of transducers. In some embodiments, the set of reflected signals may comprise one or more pulse waves. In some embodiments, the set of reflected signals may display different signal intensities and may actively change over time depending on characteristics of the underlying anatomical structures. In some cases, the magnitudes of the reflected signals may be indicative of the signal intensities. The set of reflected signals may be used to identify a location of the target site while the device 100 is placed in situ on the subject's body, without having to physically move the device over the portion of the subject's body to search for the target site. In some instances the target site may comprise an arteriovenous fistula, an arteriovenous graft, an arteriovenous shaft or any type of connection used for hemodialysis. The plurality of transducer elements may be selected from the group consisting of ultrasound sensors, acoustics sensors, optical sensors, pressure sensors, strain sensors, vibration sensors, pulse oximeters, and thermal sensors.

As illustrated in FIG. 2C, the set of reflected signals display different signal intensities depending on characteristics of the underlying anatomical structures. Row 1 reflected signal 130 shows a lower intensity than Row 2 reflected signal 130. This may indicate that Row 2 transducers are optimally located over the target site while the Row 1 transducers are close to the side of the target site. No signal is received from the Row 3 transducers which may indicate that they are located outside the target site.

The plurality of transducer elements can have different arrangements. In some embodiments of the device, the plurality of transducer elements is arranged in a one-dimensional longitudinal array or a two-dimensional grid array to form one or more detection channels. The two-dimensional grid array can comprise different shapes. By way of non-limiting examples, the two-dimensional grid array can comprise a rectangular array or a square array or a circular array or an elliptical array.

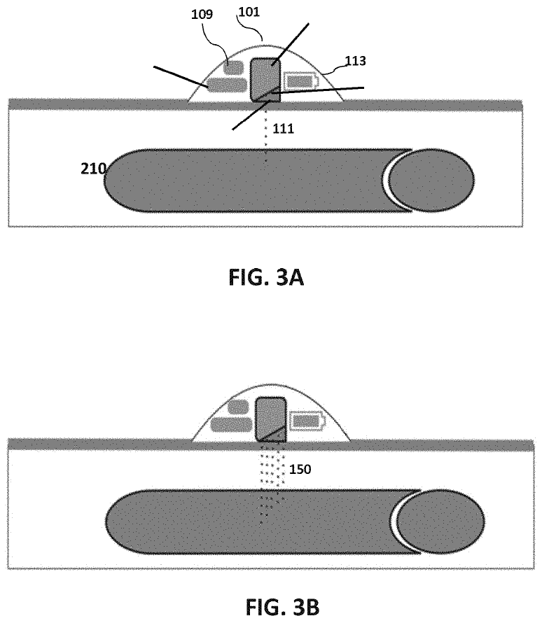

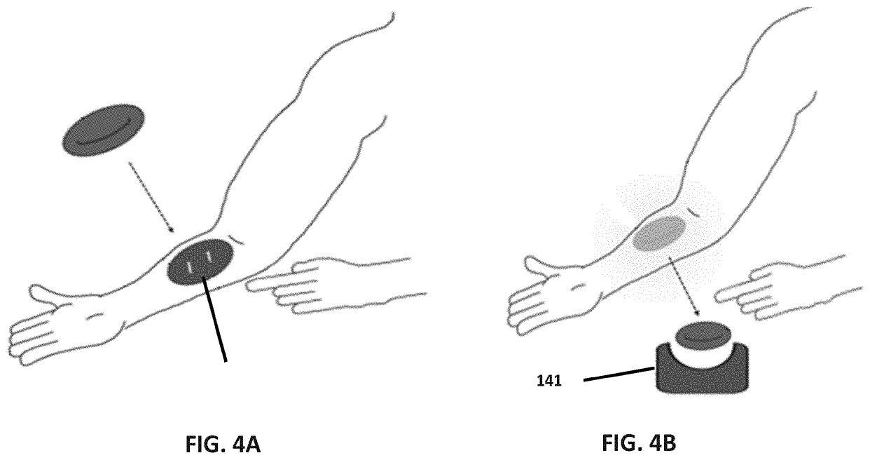

FIG. 3A illustrates the components or composition of the device 100 wherein the components may include but are not limited to the use of a transducer 111, power source 113, backing material 112, communication component 109, electrical components 108, conductive material 114 (such as but not limited to epoxy), piezoelectric element 116 (or a capacitive micromachined ultrasound transducer (CMUT)) and/or enclosure 101. The transducer in FIG. 3A and FIG. 3B may be angled to both vary and provide a fixed depth area of focus, such as the flow through a blood vessel 210. Communication component 109 can be a transmitter. In some embodiments of the device, the power source 113 may comprise a battery. The device in some examples is further configured to operably couple to a power adapter for charging the battery before or after usage of the device.

FIG. 3B illustrates an example of the components of the device 100 wherein the transducer 111 may be arranged in a multiple transducer array or multi-array arrangement 150. In some embodiments, the plurality of transducer elements may be operably coupled to a processor via wired or wireless communications. The processor may be a part of device 100 or separate from it. In some embodiments, the device 100 further may comprise the processor. In some embodiments, the processor may be configured to generate a set of image data from set of reflected signals. In some cases the set of image data may comprise color Doppler image data, 2D ultrasound image data or any other ultrasound image data.

In some instances, the processor may be provided separate from the device. In some cases, the processor may be located on a mobile device or an external monitoring unit. In those instances of the disclosure that include processor, the processor can be configured to monitor the state of the target site based on a plurality of measurements taken over a time period, wherein the time period can be on the order of hours, days, weeks, or months. The state of the target site may comprise a maturity, function and/or dysfunction of the target site.

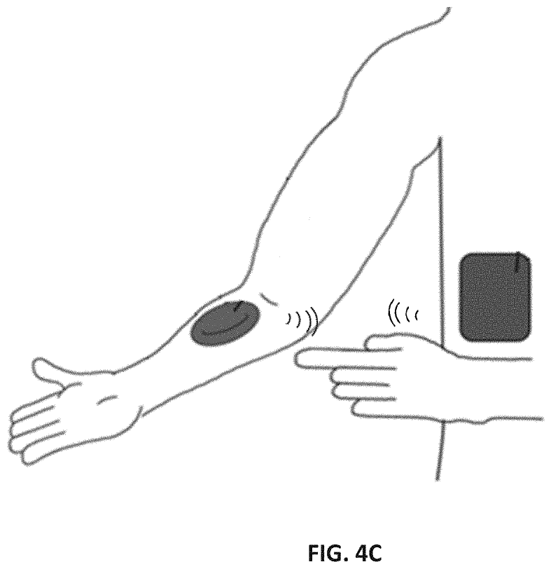

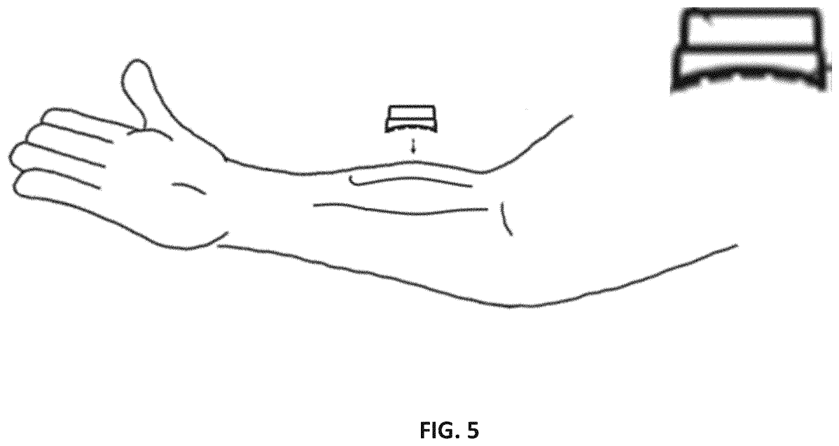

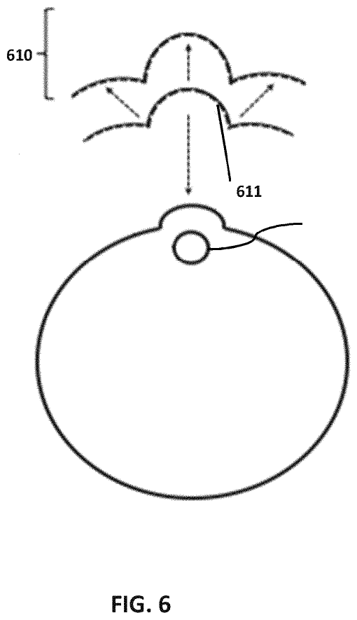

FIG. 4A illustrates an embodiment of the device 100 form factor or patient interface in which the device may be all in one and can be worn on the body. The device may be fixed in place or removable. This embodiment of the device can include a modular system and/or adaptor 140 for mounting. As shown in FIG. 4A a present embodiment of the device can include a device worn on the user's extremity that can be attached and/or removed using a suitable mechanism such as but not limited to clip, Velcro, adhesive, zipper or plastic clamp providing convenient access to the adaptor 140.

FIG. 4A and FIG. 4B illustrate embodiments of the device form factor or patient interface in which the device 100 can be intermittently placed on the skin to obtain measurements. This embodiment can provide a hub, platform, container and/or docking 141 (and/or 140) can be used to store the device 100.

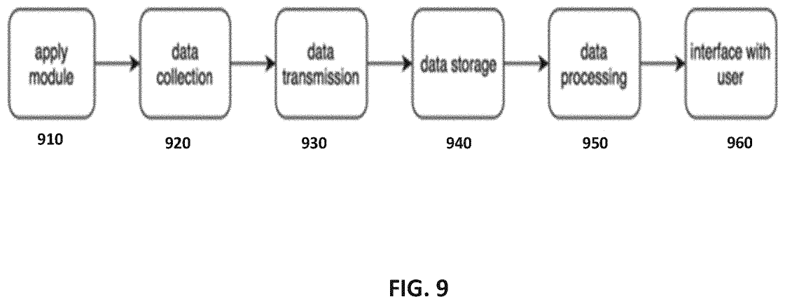

FIG. 4C illustrates an embodiment of the device form factor and/or patient interface in which the device may consist of multiple components. As shown in FIG. 4C in some embodiments of the device, the base layer can be separate from the external power source 113 and/or an external data storage device 940 (in FIG. 9) and/or an external communication device and may utilize a wired or wireless connection to communicate with any other component of the device or a user interface.

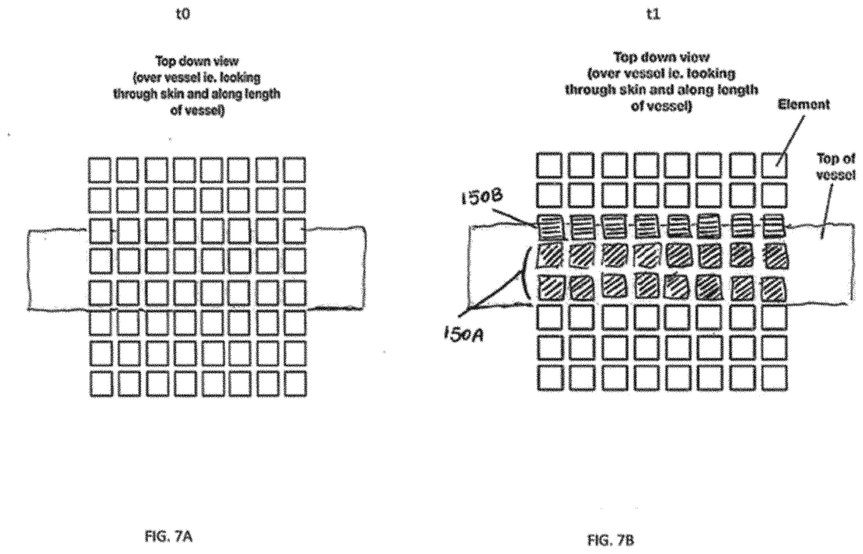

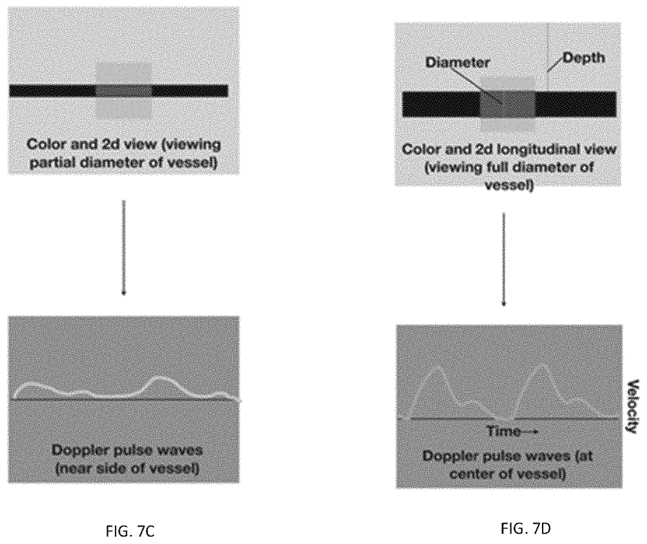

FIG. 5 and FIG. 6 illustrate embodiments of the device form factor or patient interface in which the device can be custom fit to target the measurement site(s). In some embodiments of the device, the base layer 110 can be flexible and configured to substantially conform to the portion of the subject's body. In some cases the base layer 110 may be attached to the portion of the subject's body using an adhesive, a strap, and/or a conductive medium that permits the transmitted signals and received signals to pass through. In some embodiments, the adhesives may consist of a hydrogel or may have impedance matching properties to improve signal transmission and prevent the loss of signal. In some cases, the portion of the device placed on the skin of a body site such as the forearm (e.g., around the vessel 210) to obtain measurements can be designed with mechanisms to optimize the accuracy of the measurements allowing expansion 610 and impeding compression 611 to reduce compressive risks to the patient. These mechanisms may include but are not limited to allowing for flexible, shape conforming, pressure associated, and/or pressure controlled aspects. Materials may include but are not limited to foams, synthetics, adhesives, and/or acoustic materials. These materials may be curved, ribbed, or flat.

FIG. 6 illustrates a perspective drawing of a transverse plane of a forearm wherein the embodiment of the device can be custom fit to target the measurement site 210. In some embodiments, the plurality of transducer elements may be disposed at a fixed or variable angle relative to the skin or the target site of the subject. In some cases, the angle may range from about 40 degrees to about 60 degrees. In some instances the angle may be less than 40 degrees. In other instances, the angle may be greater than 60 degrees but less 90 degrees.

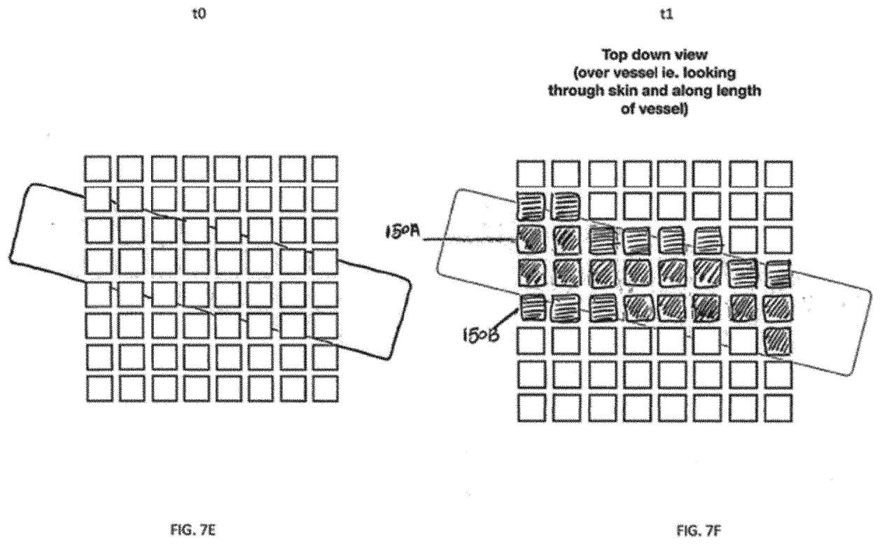

FIG. 7A shows an embodiment of the device laying over the proximity of a target site at an arbitrary time to wherein the transducer elements may not be activated or may not be transmitting any signals. FIG. 7B shows the same embodiment at an arbitrary time ti wherein the transducer elements 111 in the multi-transducer array 150 are activated and transmitting and receiving signals (for example pulse waves). The elements of transducer array 150 may be spatially configured such that the set of elements 150A (shaded squares in FIG. 7B) may lay above the target and fully cover the site and may receive the highest intensity reflected pulse waves or signals. In a similar way the set of elements 150B (horizontally filled squares in FIG. 7B) may partially cover the target site or be close to the sides of the target site in which case, the intensity of received signals may be lower than the ones in 150A. Other elements of transducer array 150 may not receive any signals. As shown in the example of FIG. 7B, in some embodiments, the first set of transducer elements having the highest reflected signal intensity may be in a straight or linear spatial configuration.

Another example of the possible spatial configuration of transducer elements in shown in FIG. 7C in which the transducer elements may not cover the full diameter of the target site or vessel. In this example the intensity of the received signal may not be optimal. In FIG. 7D an example of an embodiment is shown wherein the transducer elements may be optimally located above the center of the target site and may receive optimal high intensity pulse waves as shown in the Doppler image data.

FIGS. 7E and 7F show an example of an embodiment wherein the transducer elements and the target site may not be linearly aligned. FIG. 7E shows an embodiment of the device laying over the proximity of a target site at an arbitrary time to wherein the transducer elements may not be activated or may not be transmitting any signals. FIG. 7F shows the same embodiment at an arbitrary time ti wherein the transducer elements 111 in the multi-transducer array 150 are activated and transmitting and receiving pulse waves. The elements of transducer array 150 may be spatially configured such that the set of elements 150A (shaded squares in FIG. 7B) may lay above the target and fully cover the site and may receive the highest intensity reflected pulse waves or signals. In a similar way, the set of elements 150B (horizontally filled squares in FIG. 7F) may partially cover the target site or be close to the sides of the target site in which case, the intensity of received signals may be lower than the ones in 150A. Other elements of transducer array 150 may not receive any signals. As shown in the example of FIG. 7F, in some embodiments, the first set of transducer elements having the highest reflected signal intensity may be in a staggered or non-linear spatial configuration.

The plurality of transducer elements 111 can be spaced apart by a distance ranging from micron level to millimeter level. In some cases, the distance may be a few microns for example between 1 and 5 microns or 5 to 10 microns. In other cases, the distance may be in the order of tens of microns, for example 10 to 50 microns or 50 to less than 100 microns. In some cases, the distance may be in the order of hundreds of microns, for example 100 to 500 microns or 500 to less than 1000 microns. In some cases, the distance may be between one millimeter and less than 10 millimeters. In other cases. the distance may be less than one micron or there may be no distance between the plurality of transducer elements.

In some embodiments, the plurality of transducer elements may be spaced laterally apart by a first distance ranging from micron level to millimeter level, and spaced longitudinally apart by a second distance ranging from micron level to millimeter level. In some cases the first and/or second distance may be a few microns for example between 1 and 5 microns or 5 to 10 microns. In other cases, the first and/or second distance may be in the order of tens of microns, for example 10 to 50 microns or 50 to less than 100 microns. In some cases, the first and/or second distance may be in the order of hundreds of microns, for example 100 to 500 microns or 500 to less than 1000 microns. In some cases, the first and/or second distance may be between one millimeter and less than 10 millimeters. In other cases, the first and/or second distance may be less than one micron or there may be no first and/or second distance between the plurality of transducer elements.

In some instances, the two-dimensional grid array may comprise an m by n array, wherein m and n can be same or different integers. At least one of m or n can be greater than 2. In some embodiments of the device, the processor may be configured to receive the set of reflected signals from the plurality of transducer elements. In those embodiments, the processor may be configured to identify the location of the target site by comparing the different signal intensities within the set of reflected signals. In some cases, the processor may be configured to identify the location of the target site based on a spatial configuration of a first set of transducer elements having the highest reflected signal intensity. In other examples, the processor may be configured to use the first set of transducer elements to further monitor a state of the target site.

As described in FIG. 7 and elsewhere herein, the plurality of transducer elements may comprise (1) the first set of transducer elements and (2) a second set of transducer elements having lower reflected signal intensities than the first set of transducer elements. In some embodiments of the device, the processor may not be configured to use any of the reflected signals from the second set of transducer elements when monitoring the state of the target site. In some other instances, the processor may be configured to discard or disregard the reflected signals from the second set of transducer elements after the location of the target site has been identified. In some examples, the processor may be configured to deactivate the second set of transducer elements once the location of the target site has been identified. In some embodiments, the second set of transducer elements may be configured to (1) cease transmitting signals and (2) cease receiving reflected signals once the location of the target site has been identified.

One aspect of the present disclosure may comprise a method of locating a subcutaneous target site, the method comprising, attaching a device to a portion of the subject's body in proximity to the target site, wherein the device comprises a base layer and a plurality of transducer elements on the base layer; using the plurality of transducer elements to (1) transmit a set of signals that penetrate beneath the skin of the subject and (2) receive a set of reflected signals associated with underlying anatomical structures, the underlying anatomical structures comprising the target site and bodily tissue surrounding the target site; and analyzing the set of reflected signals to identify a location of the target site while the device is placed in situ on the subject's body, without having to physically move the device over the portion of the subject's body to search for the target site.

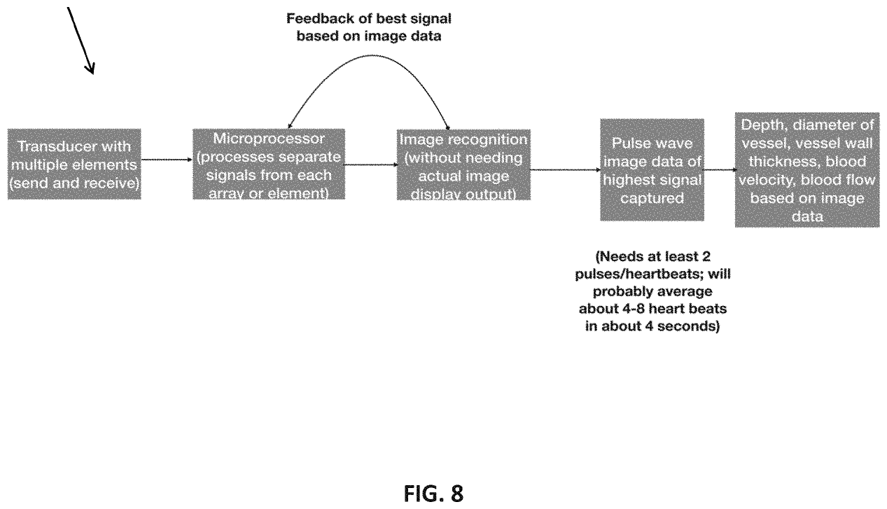

FIG. 8 shows the block diagram of method 800 for capturing the data and processing the image data. In one embodiment, multiple transducer elements may send and receive signals from the proximity of the target site. The processor can then process data from each transducer element 111 separately. The next step may consist of image recognition using the processed reflected signals. In some embodiments of the device, there may be no need for an image display or an image output, although the image can be used in the data processing and recognition. In some embodiments, the processor may be configured to generate image data of the one or more pulse waves. In the image recognition step, the feedback of the best signal based on the image data may be used to determine the best image over time. In the next step, the pulse wave image data of the highest intensity signal (optimal signal) may be captured. In some cases, the image data may be based on at least two pulses/heartbeats. In some instances, the image data may comprise an average velocity over about 4 to 8 heartbeats in about 4 seconds.

The state of the target site may be determined based on the image data. In some embodiments, the state of target site may be indicated by one or more physiological parameters selected from the group consisting of blood flow within vessel, depth from surface of the skin to the vessel wall, diameter of the vessel, velocity of blood within vessel, thickness of vessel wall and increase of diameter of the wall during the pulse or compliance of vessel. In some embodiments, the one or more physiological parameters may be associated with an arteriovenous fistula, an arteriovenous graft, an arteriovenous shunt, any type of connection used for hemodialysis, veins, arteries, capillaries, ducts, or any other anatomical conduits.

FIG. 9 illustrates an embodiment of the monitoring system wherein the system may include one or more data acquisition 920 and storage devices 940 for wired or wireless data transfer 930, including but not limited to Bluetooth, cellular and/or wireless local area networking. For example, FIG. 9 illustrates an embodiment of the monitoring system wherein the data storage device 940 can include but is not limited to metadata. Another embodiment of the monitoring system may include a data storage 940 and acquisition device 920 (and/or application 910) that can transmit data 930 to a remote system which may provide robust and/or temporary data storage 940 and processing 950. This embodiment of the monitoring system can involve healthcare professionals evaluating patient data and/or obtaining clinical guidance 960. In addition, patient data can be transferred at any step including data acquisition 920, data transmission 930, data storage 940, and/or data processing 950. All data can be transferred to interface with the user 960.

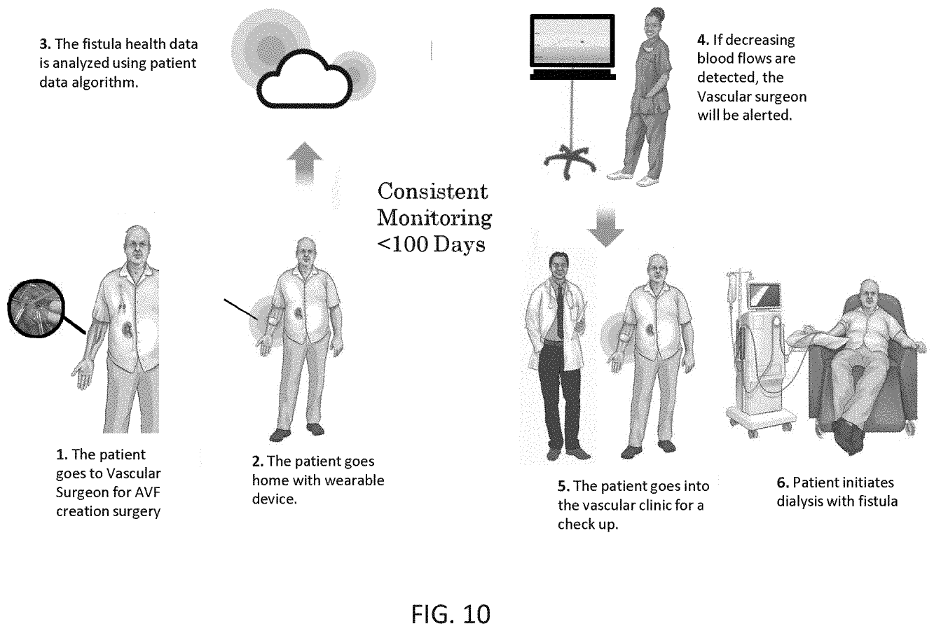

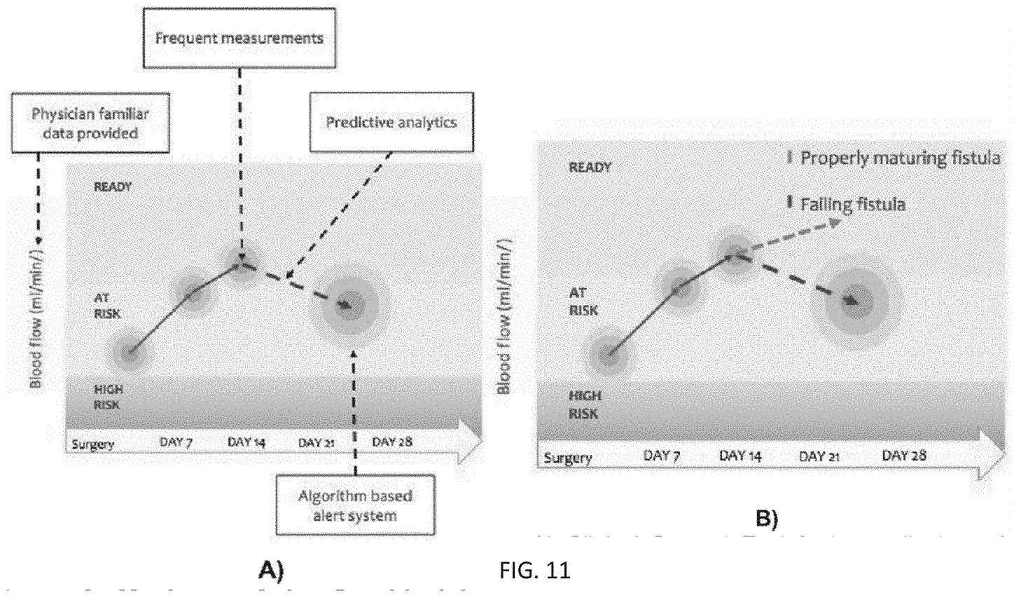

FIG. 10 illustrates a schematic of a system in which the embodiments of present disclosure may be used. As shown in FIG. 10, device 100 is placed on the surface of the body of the patient in proximity to the target site. In some embodiments the target site may comprise a surgically created subcutaneous arteriovenous fistula. Device 100, can locate and monitor the target site for any physiological changes. In some embodiments of the present disclosure, the target site may be capable of being located using the device 100, and without requiring a technician to obtain ultrasound image scans and use digital calipers on the displayed image scans to measure and find the target site. The processor of device 100 may process separate signals from each transducer element on the base layer of the device. The processor data can then analyzed using a patient data algorithm. Data processing can be done on the device or on a remote server such as a cloud. In some embodiments of the device, the processor may be configured to monitor the state of the target site based on a plurality of measurements taken over a time period. This time period can be in the order of hours, days, weeks, or months. A decision can be made based on the processed data images (described in detail in FIG. 11) and the clinician can be alerted in case of detection of a dysfunction or failure in the target site. In some embodiments, the processor may be configured to generate one or more intervention alerts to a healthcare provider or the subject (patient), based on the state of the target site. In some cases, the one or more intervention alerts may be designed to allow the healthcare provider or the subject (patient) to take corrective actions to further address a potential adverse occurrence at the target site. As illustrated in FIG. 10, the patient can go for clinical checkups while wearing the device 100. Dialysis can be initiated when the function of the access (target) site is secured using the data and image analysis of the embodiments of the present disclosure.

FIG. 11 illustrates an example of the blood flow measurements at a target site for example at an AV fistula over a period of time. Prediction analysis for failure/dysfunction or maturation/function of the target site can be done. Plurality of measurements may be made and each signal from each transducer element may be processed separately by the processor. In some embodiments, the plurality of measurements may comprise a baseline measurement and a series of interval measurements. In some embodiments, the processor may be configured to measure the maturity, function and/or dysfunction of the target site, by comparing the series of interval measurements to the baseline measurement. The maturity, function, and/or dysfunction may be measured based at least on blood flow volume, diameter of blood vessel, or depth of the vessel.

As shown in FIG. 11, in some embodiments, the maturity, function and/or dysfunction of the target site is measured based at least on blood flow volume, diameter of blood vessel, or depth of the vessel. In some embodiments, the processor may be configured to determine whether the target site is (1) progressing towards maturation or (2) digressing from maturation, based on the comparisons between the series of interval measurements to the baseline measurement. FIG. 11 is an example of a user interface, wherein some analysis may be displayed based on measurements. FIG. 11B shows two possible predictive outcomes based on the measurements. The upward pointing dotted arrow shows the case in which the target site is properly maturing or progressing towards maturation and the downward arrow shows the case wherein the target site may be failing or digressing from maturation. In this case, the processor may be configured to generate one or more intervention alerts to a healthcare provider or the patient, based on the state of the target site. Dysfunction and/or failure of the target site may occur at any time even after the maturation of the fistula. In some embodiments, the processor can be configured to determine whether the target site is (1) maintaining function when the target site is mature or (2) digressing from function which is indicative of the target site failing, based on the comparisons between the series of interval measurements to the baseline measurement.

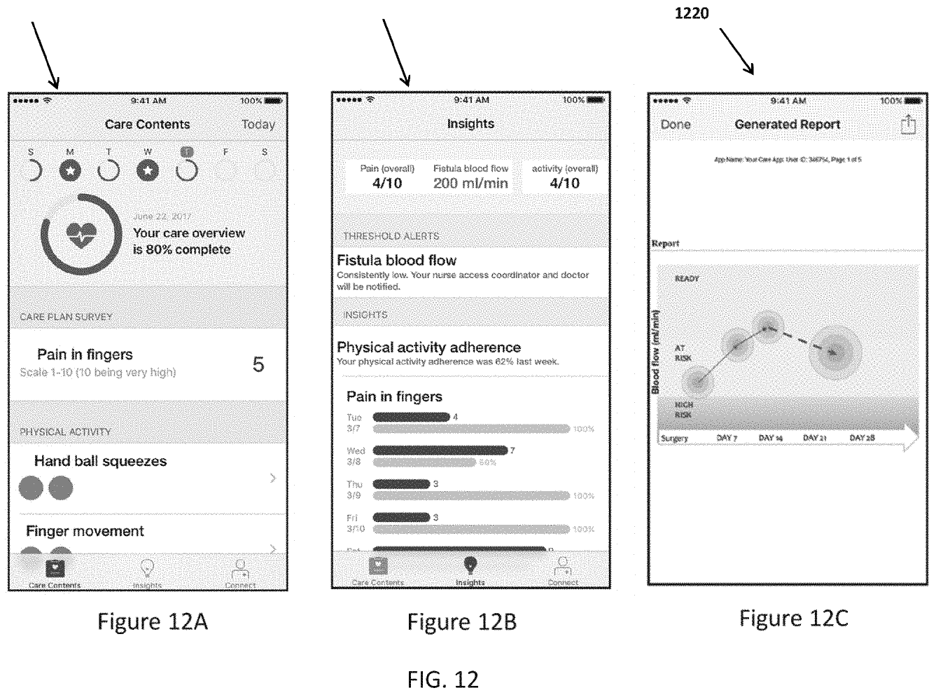

FIGS. 12A-12C show examples of a graphical user interface (GUI). A non-limiting example of a GUI is a toolkit on a smartphone enabling interaction with the patient for purpose of for example including the patient in a study or motivating the patient to comply with the care team requirements. User may enter data of some activity or a parameter. For example window 1200 shows that the patient has entered a measure for physical pain in fingers, number of ball squeezes (physical activity), finger movement and some parameters for the days of the week. In some embodiments, the patient can view difference metrics or statistics related to parameters such as but not limited to physical activity and/or parameters related to the function of target site. For example, window 1210 shows metrics such as overall pain, fistula blood flow and overall activity as well as some graphics related to pain in fingers in different days of the week. Window 1220 shows an example of an embodiment wherein a report can be generated and shown based on the measurements over time. This report can be indicative of the state of the target site.

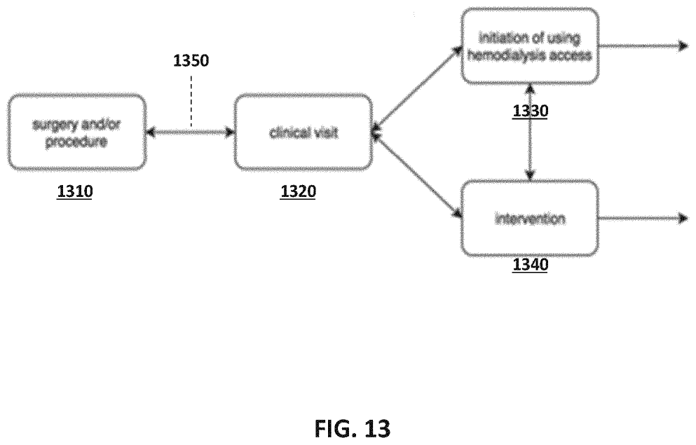

FIG. 13 shows an embodiment of the monitoring system wherein the device can be utilized at various points in the care pathway. In one example, the device can be present and/or utilized at surgery, clinical visit, during fistula maturation and/or waiting periods, dialysis, intervention and other parts of the patient care and/or physician workflow.

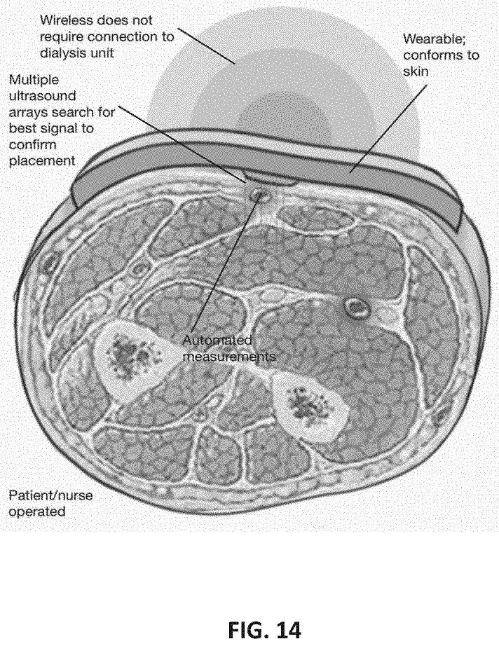

FIG. 14 shows a schematic of an embodiment of the device and illustrates some of the possible features of the device. As explained elsewhere herein, the device can be wearable and conforming to the anatomical surface where it is located. In some embodiments, multiple ultrasound arrays may search for the best or highest intensity signal to confirm placement. The device can be used in wired or wireless configurations. In some embodiments, the device may be wireless and may not need to be connected to a dialysis machine or any other machine.

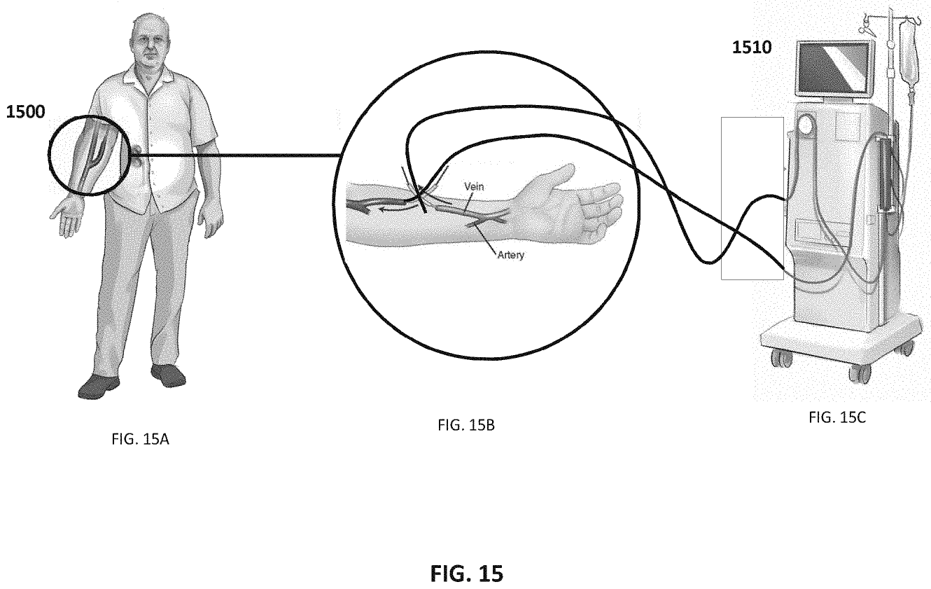

FIG. 15A shows an example of a hemodialysis access site or a target site 1500. FIG. 15B shows the subcutaneous target site 1500 in more detail. In this example the target site can be an AV fistula, where in a connection may be made between an artery and a vein. FIG. 15C shows a schematic of a hemodialysis machine and the machine's possible connections to the artery and vein through the AV fistula.

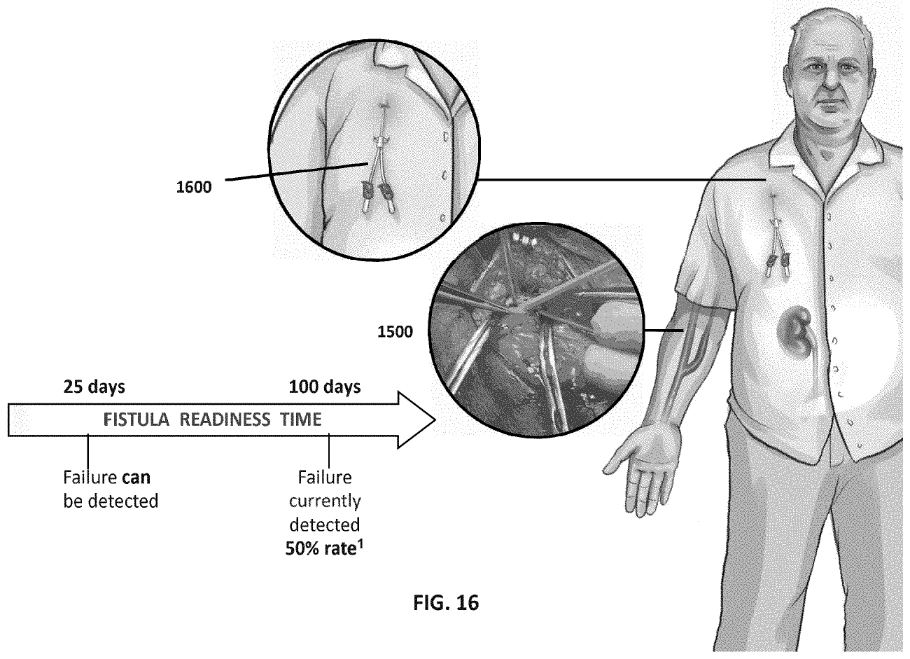

FIG. 16 shows a view of the possible procedure for creating an AV fistula surgically. A Tunneled dialysis catheter 1600 may be used in patients with a maturing fistula, but these catheters are associated with increased complications, failures and costs. A possible time frame for fistula maturation is also shown in FIG. 16. Failure may be detected as soon as for example 25 days after the creation of the target site. In some instances, the detection of failure may be possible immediately after the creation of the target site. In some cases, the failure detection may occur more gradually for example later than 25 days after creation of the target site. In some examples, the detection of failure may occur earlier than 25 days after the creation of target site.

While exemplary embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A device for locating or monitoring a subcutaneous target site, the device comprising:
a base layer configured to attach to a portion of a subject's body in proximity to the target site; and
a plurality of transducer elements on the base layer, wherein the plurality of transducer elements are configured to (1) transmit a set of signals that penetrate beneath the skin of the subject and (2) receive a set of reflected signals associated with underlying anatomical structures, the underlying anatomical structures comprising the target site and bodily tissue surrounding the target site,
wherein the set of reflected signals is used to identify a location of the target site while the device is placed in situ on the subject's body, without having to physically move the device over the portion of the subject's body to search for the target site.

2. The device of claim 1, wherein the plurality of transducer elements are arranged in a one-dimensional longitudinal array or a two-dimensional grid array to form one or more detection channels.

3. (canceled)

4. The device of claim 1, wherein the subcutaneous target site comprises an arteriovenous fistula, an arteriovenous graft, an arteriovenous shunt, or any other type of connection used for hemodialysis.

5. (canceled)

6. The device of claim 1, wherein the set of reflected signals display different signal intensities and actively change over time depending on characteristics of the underlying anatomical structures.

7. (canceled)

8. The device of claim 6, wherein the device further comprises a processor operably coupled to the plurality of transducer elements via wired or wireless communications.

9. The device of claim 8, wherein the processor is configured to generate a set of image data from the set of reflected signals.

10. The device of claim 9, wherein the set of image data comprises color Doppler image data, 2D ultrasound image data or any other ultrasound image data.

11. (canceled)

12. The device of claim 8, wherein the processor is located on a mobile device or an external monitoring unit.

13. (canceled)

14. The device of claim 8, wherein the processor is configured to receive the set of reflected signals from the plurality of transducer elements, and wherein the processor is configured to identify the location of the target site by comparing the different signal intensities within the set of reflected signals.

15. The device of claim 14, wherein the processor is configured to identify the location of the target site based on a spatial configuration of a first set of transducer elements having the highest reflected signal intensity.

16. (canceled)

17. The device of claim 15, wherein the processor is configured to use the first set of transducer elements to further monitor a state of the target site, and wherein the state of the target site is indicated by one or more physiological parameters selected from the group consisting of blood flow within vessel, depth from surface of the skin to the vessel wall, diameter of the vessel, velocity of blood within vessel, compliance of vessel and thickness of vessel wall.

18. (canceled)

19. The device of claim 15, wherein the plurality of transducer elements comprise (1) the first set of transducer elements and (2) a second set of transducer elements having lower reflected signal intensities than the first set of transducer elements.

20-26. (canceled)

27. The device of claim 2, wherein the plurality of transducer elements are spaced apart by a distance ranging from micron level to millimeter level.

28-31. (canceled)

32. The device of claim 1, wherein the plurality of transducer elements are disposed within the base layer.

33-34. (canceled)

35. The device of claim 6, wherein the set of reflected signals comprises one or more pulse waves.

36-41. (canceled)

42. The device of claim 17, wherein the state of the target site comprises a maturity, function and/or dysfunction of the target site.

43. The device of claim 42, wherein the processor is configured to monitor the state of the target site based on a plurality of measurements taken over a time period.

44-46. (canceled)

47. The device of claim 42, wherein the maturity, function and/or dysfunction is measured based at least on blood flow volume, diameter of blood vessel, or depth of the vessel.

48-49. (canceled)

50. The device of claim 17, wherein the processor is configured to generate one or more intervention alerts to a healthcare provider or the subject, based on the state of the target site.

51-54. (canceled)

55. The device of claim 35, wherein the processor is configured to generate image data of the one or more pulse waves.

56-59. (canceled)

60. A method of locating a subcutaneous target site, the method comprising:
attaching a device to a portion of the subject's body in proximity to the target site, wherein the device comprises a base layer and a plurality of transducer elements on the base layer;
using the plurality of transducer elements to (1) transmit a set of signals that penetrate beneath the skin of the subject and (2) receive a set of reflected signals associated with underlying anatomical structures, the underlying anatomical structures comprising the target site and bodily tissue surrounding the target site; and
analyzing the set of reflected signals to identify a location of the target site while the device is placed in situ on the subject's body, without having to physically move the device over the portion of the subject's body to search for the target site.
